(19) United States
(12) Patent Application Publication
(10) Pub. No.: US 2022/0258962 A1
(43) Pub. Date: Aug. 18, 2022

(54) Cover for a Segmented Biogas Reservoir

(71) Applicant: Evoqua Water Technolgies LLC, Pittsburgh, PA (US)

(72) Inventors: Victor Cormier, Fredericton, CA (US); Darin Evans, Fredericton (CA)

(73) Assignee: Evoqua Water Technologies LLC, Pittsburgh, PA (US)

(21) Appl. No.: 17/175,598

(22) Filed: Feb. 12, 2021

(51) Int. Cl. B65D 88/42

(52) U.S. Cl. CPC B65D 88/42

(57) ABSTRACT

The segmented cover is made of juxtaposed cover segments mounted over a wastewater reservoir. Floating beams define each of these cover segments, and a floating membrane is removably mounted to the floating beams over each of the cover segments. The floating beams are enclosed in a common sealed tubular envelope, which is connected to gas-impermeable connections joining the floating beams to a wall-covering skirt and to the perimeter walls of the reservoir. Because of the sealed continuous tubular envelope enclosing the floating beams, the wall-covering skirt and the gas-impermeable connections, each segment of the cover is independently sealed from outside environment and from an adjoining segment. Biogas collection from the wastewater reservoir can be maintained even when one segment of the cover is open.

The present application claims the benefit of U.S. Provisional Application No. 62/496,217, filed Oct. 11, 2016.

### TECHNICAL FIELD

The disclosure pertains to wastewater reservoirs and more particularly it pertains to a modular cover that is configured for selectively uncovering one or more segments of a wastewater reservoir.

### BACKGROUND

Biogas such as methane, carbon dioxide and hydrogen sulfide is generated by the breakdown of organic matter in the absence of oxygen. Biogas can be produced from raw materials such as agricultural waste, manure, municipal waste, plant material or sewage. Biogas can be used as a fuel for use in a gas engine for example, to convert the energy in the gas into electricity and heat.

In the scope of the present document, biogas is collected under a gas-impermeable cover laid at the surface of a wastewater reservoir. Because biogas is generated from wastewater, a certain amount of solid residues accumulates at the bottom of the reservoir. The reservoir must be cleaned periodically to remove these solid non-biodegradable residues and to maintain gas production.

For reference purposes, the following documents represent a good inventory of the relevant technologies available in the prior art.

U.S. Pat. No. 1,674,039 issued to C. A. Glass on Jun. 19, 1928;

U.S. Pat. No. 3,079,030 issued to F. D. Moyer on Feb. 26, 1963;

U.S. Pat. No. 3,313,443 issued to H. S. Dial et al., on Apr. 11, 1967;

U.S. Pat. No. 3,516,568 issued to D. C. E. Fish on Jun. 23, 1970;

U.S. Pat. No. 3,537,267 issued to M. G. Webb on Nov. 3, 1970;

U.S. Pat. No. 3,774,799 issued to M. W. Heisterberg on Nov. 27, 1973;

U.S. Pat. No. 3,874,175 issued to R. S. Winters on Apr. 1, 1975;

U.S. Pat. No. 3,933,628 issued to F. T. Varani on Jan. 20, 1976;

U.S. Pat. No. 3,980,199 issued to W. B. Kays on Sep. 14, 1976;

U.S. Pat. No. 3,991,900 issued to N. R. Burke et al., on Nov. 16, 1976;

U.S. Pat. No. 4,139,117 issued to H. S. Dial on Feb. 13, 1979;

U.S. Pat. No. 4,476,992 issued to D. H. Gerber on Oct. 16, 1984;

U.S. Pat. No. Re 30,146 issued to H. S. Dial et al., on Nov. 13, 1979;

U.S. Pat. No. 4,181,986 issued to H. E. Aine on Jan. 8, 1980;

U.S. Pat. No. 4,230,580 issued to C. Dodson on Oct. 28, 1980;

U.S. Pat. No. 4,438,863 issued to J. V. Wilson et al., on Mar. 27, 1984;

U.S. Pat. No. 4,503,988 issued to D. H. Gerber on Mar. 12, 1985;

U.S. Pat. No. 4,672,691 issued to C. J. DeGarie et al., on Jun. 16, 1987;

U.S. Pat. No. 5,265,976 issued to J. V. Russell on Nov. 30, 1993;

U.S. Pat. No. 5,505,848 issued to R. Landine et al., on Apr. 9, 1996;

U.S. Pat. No. 5,562,759 issued to W. D. Morgan et al., on Oct. 8, 1996;

U.S. Pat. No. 5,587,080 issued to R. Landine et al., on Dec. 24, 1996;

U.S. Pat. No. 6,136,194 issued to S. M. Vogel et al., on Oct. 24, 2000;

U.S. Pat. No. 6,324,792 issued to C. J. DeGarie on Dec. 4, 2001;

U.S. Pat. No. 6,338,169 issued to C. J. DeGarie on Jan. 15, 2002;

U.S. Pat. No. 6,357,964 issued to C. J. DeGarie on Mar. 19, 2002;

U.S. Pat. No. 6,389,757 issued to C. J. DeGarie on May 21, 2002;

U.S. Pat. No. 6,451,206 issued to R. Charbonneau on Sep. 17, 2002;

U.S. Pat. No. 6,497,533 issued to C. J. DeGarie on Dec. 24, 2002;

U.S. Pat. No. 6,612,079 issued to C. J. DeGarie et al., on Sep. 2, 2003;

U.S. Pat. No. 6,851,891 issued to J. W. Baumgartner et al., on Feb. 8, '05;

U.S. Pat. No. 6,855,253 issued to J. W. Baumgartner et al., on Feb. 15, '05;

U.S. Pat. No. 6,865,754 issued to B. MacLean et al., on Mar. 15, 2005;

U.S. Pat. No. 7,309,431 issued to C. J. DeGarie on Dec. 18, 2007;

U.S. Pat. No. 7,374,059 issued to W. D. Morgan et al., May 20, 2008;

U.S. Pat. No. 7,430,834 issued to C. J. DeGarie on Oct. 7, 2008;

U.S. Pat. No. 9,249,932 issued to S. Awada et al., on Feb. 2, 2016;

CA Appl. 2,635,626 publ. by J. J. R. MacQueen et al., on Dec. 21, 2008.

Although the wastewater reservoir covers in the prior art deserve undeniable merits, none of these covers is scalable to very large reservoirs, or has relatively large openable segments for working inside large reservoirs in a safe and efficient manner Therefore, it is believed that there is a need in the industry for an improved cover to address the shortcomings of the covers found in the prior art.

### SUMMARY

In the present disclosure, there is described a segmented cover for a wastewater reservoir generating biogas. This segmented cover is scalable to very large wastewater reservoirs, and it is divisible into large openable segments. Because of this cover, a wastewater reservoir can be periodically cleaned in large areas at a time.

In a first aspect of the present segmented cover, there is provided a segmented cover for a wastewater reservoir having perimeter walls and a gas-impermeable wall-covering skirt covering inside surfaces of the perimeter walls. The cover is made of juxtaposed cover segments defined inside the perimeter walls. Floating beams define each of the cover segments. A floating membrane is removably mounted to the floating beams over each of the cover segments. The floating beams are enclosed in a common sealed tubular envelope. This envelope is connected to gas-impermeable connections joining the floating beams to the wall-covering skirt and to the perimeter walls of the reservoir. Because of the sealed continuous tubular envelope enclosing the floating beams, the wall-covering skirt and the gas-impermeable connections, each segment of the cover is independently sealed from outside environment and from adjoining segments. Biogas collection from the wastewater reservoir can be maintained even when one segment of the cover is open.

In another aspect, there is provided a segmented cover mounted over a wastewater reservoir having floating scum at the surface thereof The floating beams defining each of the cover segments are comprised of a pair of short beams and a pair of long beams; each of the long beams has a keel thereunder. When a cover segment is removed, these long beams bordering this cover segment are held laterally firm in a parallel relationship with each other by an engagement of these keels in the scum of the reservoir.

In a further aspect of the segmented cover, there is provided a method for connecting the flexible floating membrane to a biogas outlet pipe mounted fixed above the flexible floating membrane. This method comprises the steps of:
affixing a sealing plate to an opening in the flexible floating membrane;
mounting a flange to the biogas outlet pipe;
mounting lifting hooks to the sealing plate;
hoisting the lifting hooks and the sealing plate to the flange of the biogas outlet pipe, and
bolting the flange to the sealing plate.

In yet another aspect of the segmented cover, there is provided a method for fabricating a sheet joint in an accessory fastening element for a segmented cover made of thermoplastic sheet material. This method comprises the steps of:
overlapping a top layer of the sheet joint over a bottom layer of the sheet joint;
welding the top layer along an edge of the bottom layer, forming a thermoplastic weld through the top and bottom layers;
during the step of welding, leaving a longitudinal flap in the top layer extending away from the thermoplastic weld and from the edge of the bottom layer. That longitudinal flap being as wide as a fused portion of the thermoplastic weld and a work-holding margin on each side of the fused portion.

This brief summary has been provided so that the nature of the invention may be understood quickly. A more complete understanding of the invention can be obtained by reference to the following detailed description of the preferred embodiment thereof in connection with the attached drawings.

### BRIEF DESCRIPTION OF THE DRAWINGS

A preferred embodiment of the segmented cover is described with the aid of the accompanying drawings, in which like numerals denote like parts throughout the several views:

FIG. 1 is a plan view of a large wastewater reservoir that is of interest herein;

FIG. 2 is an enlarged plan view of the covered end of the wastewater reservoir illustrated in FIG. 1;

FIG. 3 is another enlarged plan view of the wastewater reservoir illustrated in FIGS. 1 and 2, showing four juxtaposed openable cover segments over the inlet end of the reservoir;

FIG. 4 is a partial perspective view of a floating beam separating segments of the segmented cover, as can be seen in detail circle 4 in FIG. 3;

FIG. 5 is a perspective view of a T-connector mounted along the beam illustrated in FIG. 4;

FIG. 6 is a perspective view of an end connector connecting any one of the floating beams of the segmented cover to the perimeter walls of the reservoir;

FIG. 7 is an isometric view of a hollow floating box mounted inside the floating beam of FIG. 4;

FIG. 8 is a general cross-section view of the floating beam of FIG. 4;

FIG. 9 is a plan view of a connection of a floating beam in the segmented cover, to the perimeter wall of the wastewater reservoir;

FIG. 10 is a partial elevation cross-section view of the attachment of the floating beam to a perimeter wall of the reservoir, as seen at line 10 in FIG. 9;

FIG. 11 is a partial cross-section view of a floating beam in the segmented cover, as seen along line 11 in FIG. 2;

FIG. 12 represents a sealed tubular envelope enclosing the floating beams in the segmented cover;

FIG. 13 is a cross-section view of a weight line attached to the segmented cover;

FIG. 14 is a cross-section view of a floating line attached to the segmented cover;

FIG. 15 illustrates a method of making a welded joint in a fastening elements of the segmented cover;

FIGS. 16-17 represent outlines of a thermoplastic welding tool used in making welded joints in the fastening elements of the segmented cover;

FIG. 18 illustrates a cross-section of a connection of a biogas outlet pipe to the floating membrane of the segmented cover;

FIG. 19 is another enlarged plan view of the inlet end of the wastewater reservoir, showing the progression of a scum formation inside one segment of the cover;

FIG. 20 is a cross-section view of a floating beam in the segmented cover, as seen along line 20 in FIG. 19.

The drawings presented herein are presented for convenience to explain the functions of all the elements included in the preferred embodiment of the segmented cover. Elements and details that are obvious to the person skilled in the art may not have been illustrated. Conceptual sketches have been used to illustrate elements that would be readily understood in the light of the present disclosure. These drawings are not fabrication drawings, and should not be scaled.

### DESCRIPTION OF THE PREFERRED EMBODIMENT

Referring firstly to FIG. 1, the wastewater reservoir 20 in the preferred embodiment is a very large reservoir having a total length “A” of 1.5 km for example. The longitudinal axis of the reservoir is oriented East-West as can be appreciated from the compass North arrow 22 illustrated in the top left corner of the reservoir 20. Because of its orientation, the reservoir is subjected to prevailing westerly winds. Therefore, wind-induced stresses on this cover is a major design concern.

The covered portion of this reservoir 20 has a length “B” of 450 meters. Because of the magnitude of the wind-induced stresses on this segmented cover and because every joint in an impermeable cover becomes a potential risk for gas leak, it will be appreciated that any openable segment therein is preferably made using a floating membrane technology. This type of design has a minimum number of joints, no raised edge to catch the wind, it is not complicated to seal, and it is removable in a single piece.

Referring to FIG. 3, the preferred segmented cover 24 is made of pieces of floating membrane 26 with associated weight lines 28 and float lines 30. The floating cover 24 is divided in segments 32 by floating beams 34. Each segment 32 is covered by an individual piece of floating membrane 26. Effluent inlet pipes 36 to the reservoir are shown on the right-hand side of FIG. 3. Biogas outlet pipes 38 are joined to biogas collection lines 40. Access ports on this cover are shown as label 42.

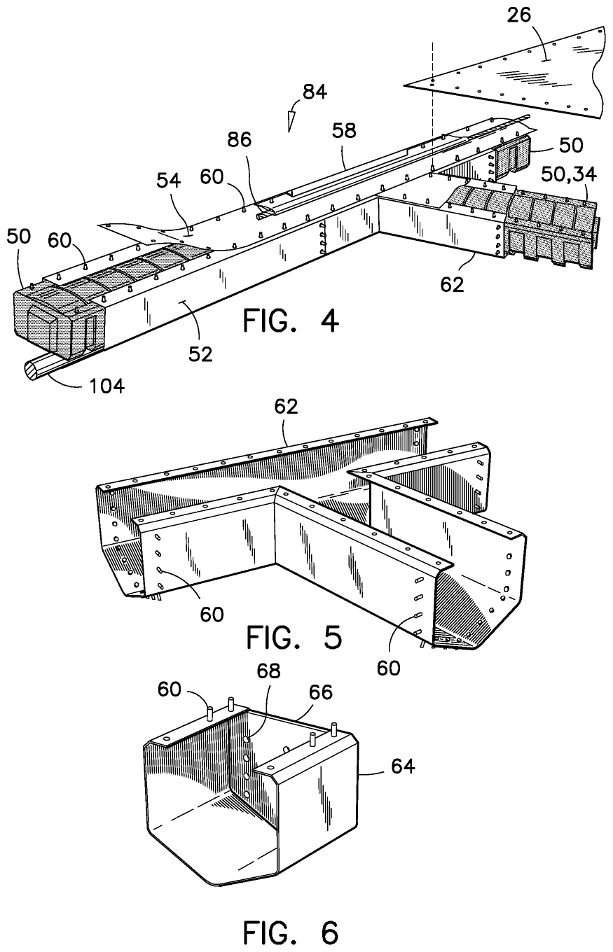

Referring now to FIGS. 4-9, the structure of the preferred floating beams 34 will be described. Each floating beam 34, is made of a chain of floating boxes 50 mounted end-to-end inside an envelope of flexible smooth-surfaced gas-impermeable sheet of High Density Polyethylene (HDPE) plastic material. This envelope of smooth-surfaced sheet material comprises a bottom wrap 52 and a top wrap 54. This envelope 52, 54 extends without join between the ends or branches of a beam 34.

As illustrated in FIG. 4, the top wrap 54 overlaps the edges of the bottom wrap 52 and is sealed to the edges of the bottom wrap 52 and to the chain of hollow boxes 50 by sealing strips, such as rubber sealing strips for example, as illustrated at label 56 in FIG. 8. The edges of both the top wrap 54, and the bottom wrap 52 are held tight in a sealed manner against the chain of hollow boxes 50 by a pair of structural angles 58. These angles 58 are bolted to the hollow boxes to threaded studs 60 extending from the top of each box 50.

Each bottom wrap 52 and each top wrap 54 are sealed to outside surfaces of branch connectors 62, and end connectors 64 using similar threaded studs 60 in these connectors and stiff flat bars (not shown) having a same clamping function as the angles 58. The branch connectors 62 and the end connectors 64 are made of stainless steel or aluminum plates. The connectors have dimensions to loosely encapsulate a portion of a hollow box 50.

Each end connectors 64 has an end plate 66 that is preferably made of a stronger material than the sides, and that end plate 66 has holes 68 therein to receive anchor bolts 70, as illustrated in FIG. 10, to retain the end connector 64 to a concrete wall 72 of the reservoir. Preferably, the structural angles 58 in each beam 34 are also anchored to the wall 72 of the reservoir, as can be seen in FIGS. 8, 9 and 10.

Each piece of floating membrane 26 is fastened to the floating beams 34, to the threaded studs 60, and are retained in a sealed manner to the beams by the sealing strips 56 and structural angles 58, as it can be seen in FIGS. 4, 8 and 9.

Each piece of floating membrane 26 is also made of flexible High Density Polyethylene (HDPE) plastic sheet material. Each piece of floating membrane 26 is also fastened and sealed to the wall 72 of the reservoir in a similar manner as the beam envelope mentioned before. It will be appreciated that each piece of floating membrane 26 is removable from any one segment 32 by removing the angles 58 and by rolling the piece of membrane 26 toward one end of the segment 32.

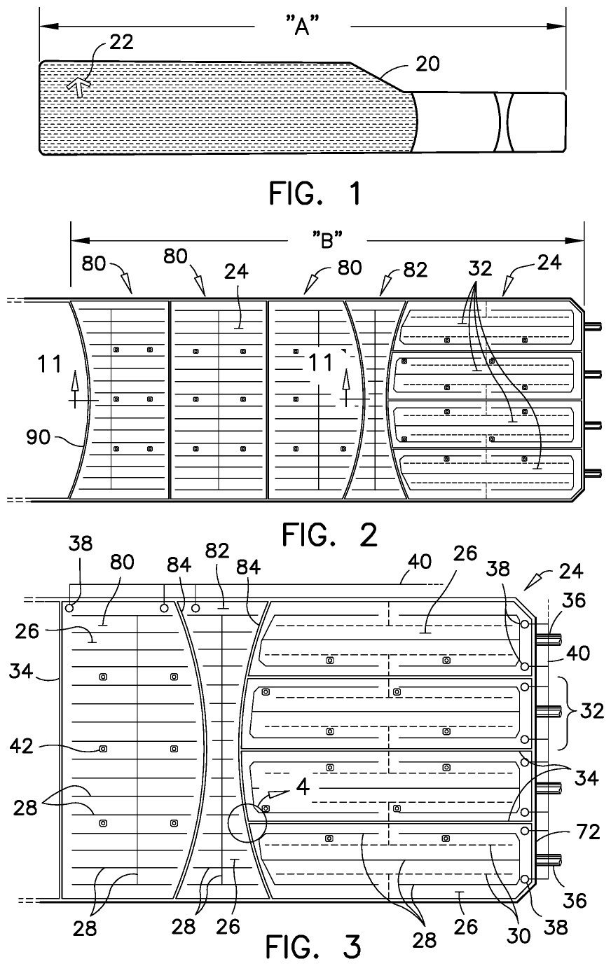

Referring back to FIGS. 2 and 3, the preferred segmented cover 24 has four longitudinal segments 32 over a region of high gas production, near the effluent discharge pipes 36 of the reservoir. The segmented cover 24 also has three transverse segments 80 covering a lower gas production area, adjacent to the longitudinal segments 32. The longitudinal segments 32 are separated from the traverse segments 80 by a prestressed segment 82. This prestressed segment 82 comprised of two spaced-apart arcuate beams 84 oppositely arced in a catenary shape and extending across the width of the reservoir. The space between the two beams 84 is sealed by a piece of flexible floating membrane 26. The arcuate floating beams 84, and the straight floating beams 34 constitute floating walls, defining and dividing the segments of the segmented cover 24.

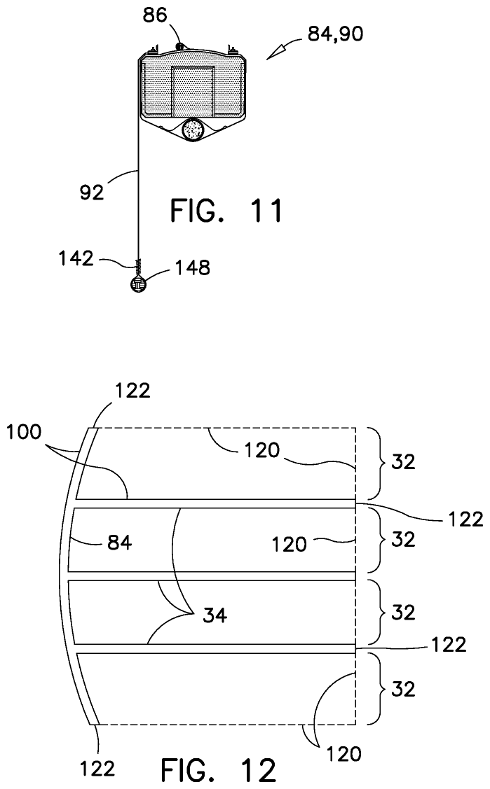

The prestressed segment 82 extends across the prevailing winds on the cover, to strengthen the segmented cover. Each arcuate beam 84 in the prestressed segment 82 has a cable 86 fastened along its top surface, as can be seen in FIGS. 4 and 8. These cables 86 are anchored to solid structure (not shown) outside the reservoir. These cables 86 maintain a tension force along the arcuate floating beams 84 to resists wind loads on the cover and to provide a solid support to the longitudinal segments 32. The outmost floating beam 90 of the segmented cover is also a prestressed arcuate floating beam as just explained. Preferably, the outmost floating beam 90 and the outmost floating beam 84 in the prestressed segment 82, have skirting 92 there-along as illustrated in FIG. 11, for keeping biogas under the covered portion 24 of the reservoir.

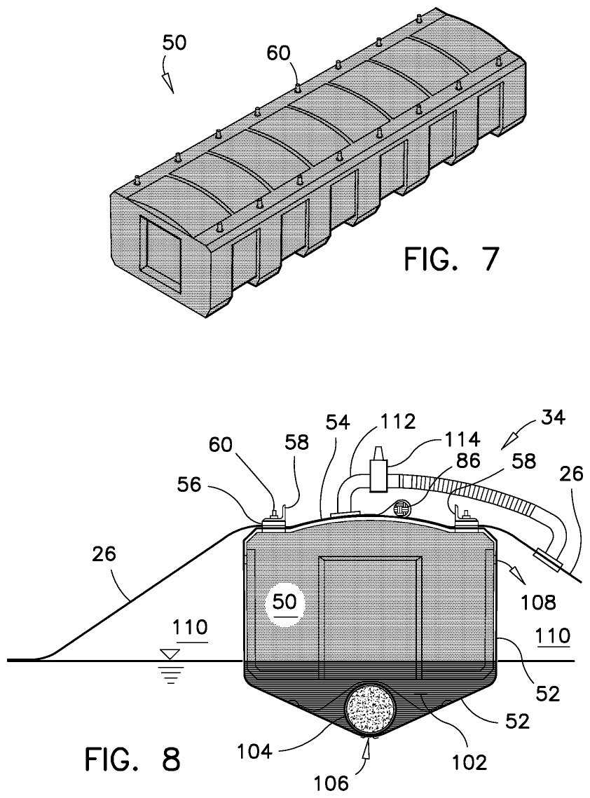

Each floating box 50 has a boss at one end and a socket at the other end, as can be appreciated from FIGS. 4 and 7. The boss of one box fits into the socket of an adjacent box in a chain of floating boxes. When a chain of floating boxes is held together by the cable 86, the alignment of the chain is maintained by the engagement of these bosses and sockets.

Referring now to FIG. 8, the tubular envelope defined by the bottom wraps 52 and a top wraps 54 encapsulating the floating beams 34 and connectors 62, 64, constitute a gas-impermeable barrier network 100 as represented in FIG. 12. This gas-impermeable barrier network 100 is ballasted with wastewater 102 from the reservoir and by a weight pipe 104. For that purpose, openings 106 are made in the lower portion of the bottom wrap 52 at spaced intervals. Because the wastewater 102 in the gas-impermeable barrier network 100 is susceptible of generating biogas, additional gas openings in a region shown at arrow 108 are made in the upper portion of the bottom wrap 52 at spaced intervals.

The gas openings at arrow 108 communicate with the biogas accumulation region 110 alongside each beam 34. For more certainty, to prevent inflation of the gas-impermeable barrier network 100, one or more gas vents 112 and isolation valves 114 are installed in the top wrap 54 of the tubular envelope. These vents 112 are connected to biogas accumulation regions 110 under the floating membranes 26.

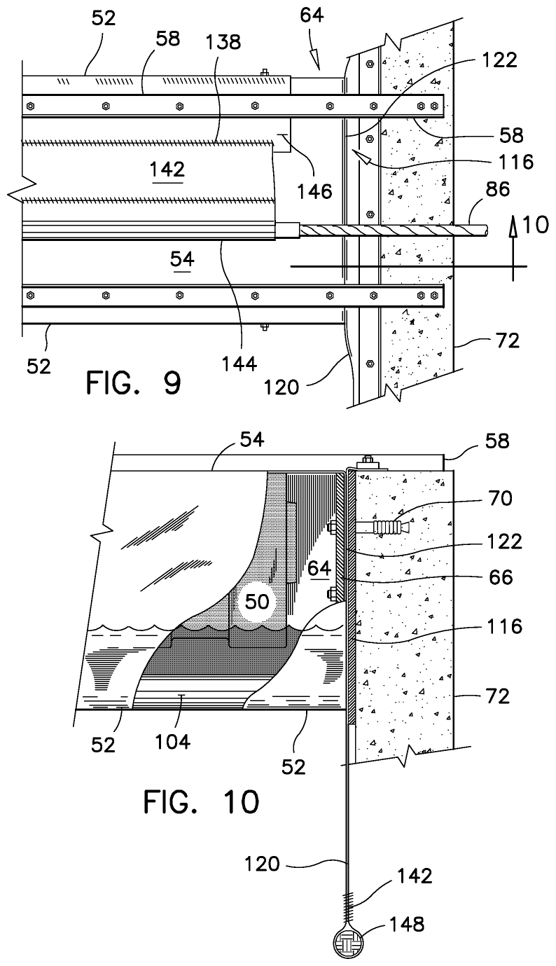

Referring to FIGS. 9 and 10, the end connector 64 on a floating beam 34 is sealed to a wall 72 of the reservoir, by way of an adapter plate 116. The adapter plate 116 is fitted to the wall 72 of the reservoir with grout for example to provide a seal joint between the adapter plate 116 and the wall 72 of the reservoir.

The end plate 66 of the end connector 64 is bolted to anchor bolts 118 extending into the wall 72 of the reservoir, for retaining the end connector 64 and the adapter plate 116 tight against the wall 72 of the reservoir. Prior to tightening the anchor bolts 118, the bottom wrap 52 and the top wrap 54 of the tubular envelope are folded over the end connector 64 and clamped in a sealed manner between the end plate 66 and the adapter plate 116. The adapter plate 116 provides a flat surface against which the folds of the bottom wrap 52 and top wrap 54 can be clamped tight and sealed.

Referring especially to FIG. 10, a gas-impermeable wall-covering skirt 120 is installed along the concrete wall 72 of the reservoir. This gas-impermeable wall-covering skirt 120 is also sealed between the end plate 66 of the end connector 64 and the adapter plate 116. As can be appreciated, the end joint 122 between a beam 34 and the wall 72 of the reservoir is a gas-impermeable connection.

Because of the gas-impermeable barrier network 100; the gas

[symbol]

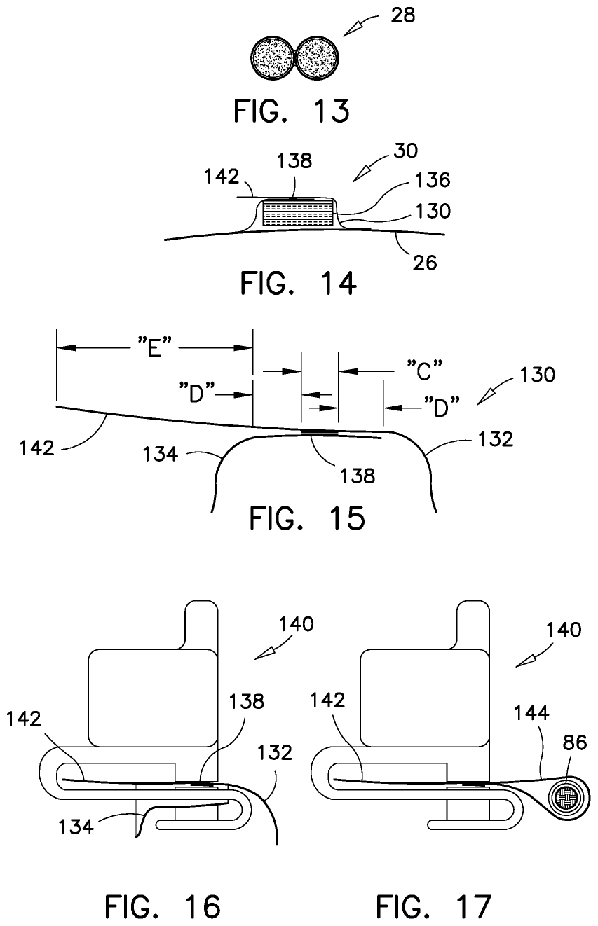

Referring to FIG. 13, the weight lines 28 are made of two lengths of pipes welded parallel together and filled with grout. This welded arrangement is convenient as the weight lines 28 are less susceptible to move out of place during the inflation of the cover. The purpose of the weight lines 28 and the float lines 30 is to create depressions and ridges on the cover to collect rainwater, and to promote the migration of biogas toward the biogas collection pipes 38. Further explanation as to where rainwater collects or about locations of rainwater pumps and gas migration pathways is not provided as this is well known in the art.

A float line 30 is attached to a piece of floating membrane 26 inside a channel 130 that is welded to the top surface of the floating membrane 26, as illustrated in FIG. 14. This channel 130 is preferably fabricated from two strips 132, 134 of high density polyethylene plastic sheet material, as it may be understood from FIG. 15. These two strips 132, 134 previously attached to the membrane 26, are folded over a float element 136 and fused by thermoplastic weld over the float element 136. The fused portion of the weld is represented by label 138, as seen in FIGS. 14 and 15.

The thermoplastic welds are preferably made using a deep throat welder 140 as illustrated in FIGS. 16 and 17, and a weld placement method as explained in FIG. 15. The preferred weld placement method consists of creating an overlap of the strips 132, 134 in such a way as to cover the width “C” of the fused portion 138 of the weld, the widths “D” of a work holding margin on each side of the fused portion, and a loose flap 142 of width “E” of about the same width or more than the dimension “C” plus two times the dimension “D”. This loose flap 142 is convenient for attaching other element of the cover thereto.

An example of the advantages of this weld placement method may be understood from the illustrations of FIGS. 9 and 16. In FIG. 9, the cable 86 holding the chain of floating boxes together is retained to the top of the floating boxes by a fastening sleeve 144 of HDPE plastic sheet. When this fastening sleeve 144 has an extended flap 142 as mentioned before, it can be easily affixed to another flap 146 previously affixed to the top wrap 54 for example, as illustrated in FIG. 9. Similarly, a belt holding the weight lines can be fastened to the flap of a float line, etc. Other examples are illustrated in FIGS. 10 and 11. A sleeve enclosing a weight tube 148 hung to skirt 92 or 120 is similarly attached to the skirt by an extended flap 142. Such extended flap 142 facilitates greatly the fabrication of a floating cover in the field.

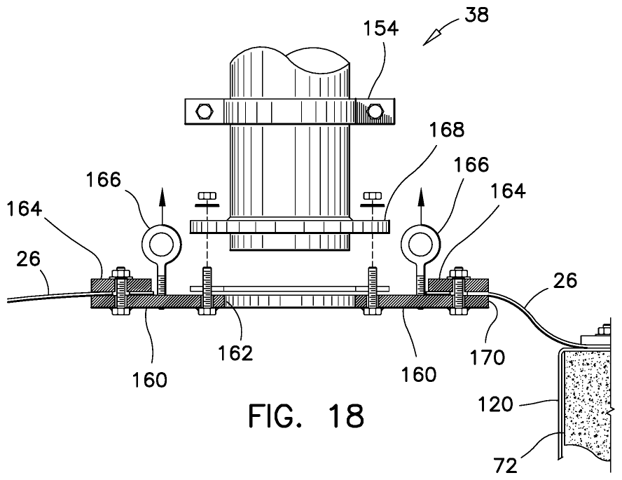

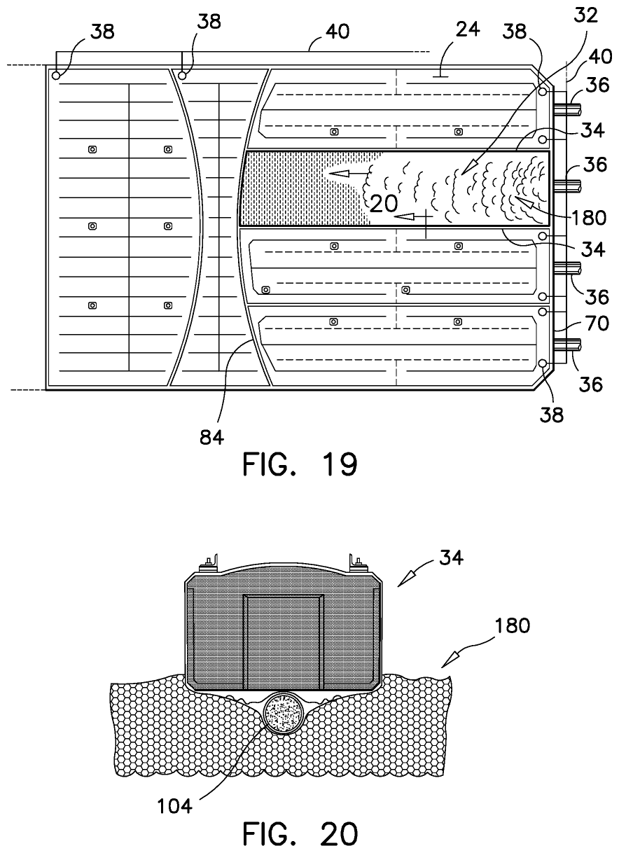

Referring now to FIG. 18, another characteristic of the segmented cover will be explained. When a specific segment of the cover is removed, its respective floating membrane 26 must be disconnected from the gas outlet pipes 38 associated with that segment. These connections must be redone when the cover segment is reinstalled.

While the disconnecting of the gas outlet pipe 38 may be done relatively easily, the reinstallation is a challenge. This gas outlet pipe 38 is normally fixed to a rigid structure 154 and cannot be moved down toward the floating membrane 26. The floating membrane 26 must be brought upward to connect to the end of the pipe 38. It is unsafe, unpleasant and unpractical for a worker to go under the cover, in a dinghy, a raft or otherwise, to push the membrane 26 upward and to reconnect the floating membrane 26 to the gas outlet pipe 38.

In the preferred embodiment, the floating membrane 26 is fitted with a sealing plate 160 having a gas outlet hole 162 there-through. The sealing plate 160 can be installed to the floating membrane 26 by sliding it under the floating membrane 26 from the wall 72 of the reservoir for example. A sealing disc 164 is attached to the top of the sealing plate 160. The sealing disc 164 is installed in such a way as to seal the floating membrane 26 to the top surface of the sealing plate 160.

A pair of eye bolts 166 are affixed to the sealing plate 160. The sealing plate 160 with the floating membrane 26 is brought up by portable hoists (not shown) engaged to the eye bolts 166. The sealing plate 160 is maneuvered while suspended to the hoists and aligned with the gas outlet pipe 38. A flange 168 on the gas outlet pipe 38 can then be bolted to the sealing plate 160, to secure the floating membrane 26 to the gas outlet pipe 38. It will be appreciated that the edge 170 of the sealing plate 160 along the wall 72 of the reservoir is a straight edge. This straight edge 170 is aligned parallel to the wall 72 of the reservoir so as to prevent the formation of wrinkle in the floating membrane 26. Because of this sealing plate arrangement, the making of a connection or the undoing of that connection is made by personnel standing on the upper side of the floating membrane 26, with minimum exposure to the conditions under the floating membrane.

In yet another characteristic of the preferred segmented cover 24 is illustrated in FIGS. 19 and 20. The second segment 32 from the top of the illustration is shown with its floating membrane 26 removed. In a typical wastewater reservoir, scum 180 tend to accumulate near the effluent discharge pipes 36. Typically, this scum 180 forms a thick, wax-like floating mat over the surface of the reservoir. The floating mat becomes thicker near the discharge pipes 36 and moves slowly as a glacier toward the far end of the reservoir.

Opening and cleaning of one segment 32 of the reservoir is generally done when the scum mat 180 reaches the far end of a segment 32. At that time, the layer of scum 180 entraps the floating beams 34 therein, as represented in FIG. 19, with the weight pipe 104 in each beam 34 acting as a keel or a key, to lock each beam 34 against lateral movement.

As explained before, each floating beam 34 bordering one segment 32 is attached at one end to the concrete wall 72 of the reservoir, and at the other end to one of the arcuate beams 84, which is by its nature strong and rigid. The side floating beams 34 are keyed to the layer of scum 180. Consequently, the entire perimeter of an open segment 32 is stiff and resistant to lateral deflection. Personnel can safely walk or travel by small vehicle on the cover around the open segment 32 for the purpose of installing a barge and backhoe for example to clean the scum and solid residues out of that open segment of the reservoir.

What is claimed is:

1. A segmented cover for a wastewater reservoir comprising:
perimeter walls and a gas-impermeable wall-covering skirt covering inside surfaces of said perimeter walls;
juxtaposed cover segments defined inside said perimeter walls;
floating beams defining each of said cover segments, and
a floating membrane removably mounted to said floating beams over each of said cover segments;
said floating beams being enclosed in a common sealed tubular envelope; said tubular envelope being connected to gas-impermeable connections joining said floating beams to said wall-covering skirt and to said perimeter walls.

2. A segmented cover for a wastewater reservoir comprising:
juxtaposed cover segments;
floating beams defining each of said cover segments, and
a floating membrane removably mounted to said floating beams over each of said cover segments;
each of said floating beams comprising floating boxes mounted end to end;
each of said floating beams being wrapped in a sealed tubular envelope and all of said tubular envelopes being joined together defining a sealed gas-passage network; and
said gas passage network being ballasted with wastewater from said wastewater reservoir, and being open to a gas accumulation region under one of said floating membranes.

3. A wastewater reservoir having floating scum at the surface thereof
and a segmented cover mounted there-over, said segmented cover comprising:
juxtaposed cover segments;
floating beams defining each of said cover segments, and
a floating membrane removably mounted to said floating beams over each of said cover segments;
said floating beams defining each of said cover segments comprising a pair of short beams and a pair of long beams;
each of said long beams having a keel thereunder;
said long beams being held laterally firm in a parallel relationship with each other by an engagement of said keels in said scum.

4. The wastewater reservoir as claimed in claim 3, further comprising a pair of oppositely horizontally arcuate floating beams joined together by one of said floating membranes, and one of said short beams defining each of said cover segments is comprised in one of said arcuate floating beams.

5. The wastewater reservoir as claimed in claim 3, wherein each of said floating beams comprises a plurality of floating boxes joined together end-to-end.

6. The wastewater reservoir as claimed in claim 5, wherein said floating beams comprises a cable mounted thereto for firmly holding said floating boxes end-to-end.

7. The wastewater reservoir as claimed in claim 5, wherein said floating boxes in each of said floating beams are wrapped in a flexible, smooth-surfaced, impermeable sheet defining a sealed tubular envelope around said each of said floating beams, and all of said envelopes being joined together.

8. The wastewater reservoir as claimed in claim 5, further comprising a concrete wall extending there-around, and each of said long beams being connected to one of said concrete walls by a steel box bolted to said concrete wall and partly enclosing one of said floating boxes.

9. The wastewater reservoir as claimed in claim 8, wherein each of said long beams being connected to one of said arcuate beams by a metal box partly enclosing one of said floating boxes in said long beam and partly enclosing two of said floating boxes in said arcuate beam.

10. The wastewater reservoir as claimed in claim 9, wherein each of said floating boxes comprises threaded studs extending upward, and each of said floating membranes is removably affixed to said threaded studs.

11. The wastewater reservoir as claimed in claim 7, wherein said tubular envelopes being partly filed with wastewater.

12. The wastewater reservoir as claimed in claim 7, wherein said tubular envelope comprises a longitudinal sheet overlap and a thermoplastic weld in said sheet overlap; said thermoplastic weld comprising a fused portion and a work-holding margin on each side of said fused portion, and said sheet overlap also comprising a longitudinal flap bordering said thermoplastic weld, and a width of said longitudinal flap being as much as a width of said thermoplastic weld.

13. A method for installing a flexible floating membrane over a wastewater reservoir and for connecting said flexible floating membrane to a biogas outlet pipe mounted fixed above said flexible floating membrane, comprising the steps of:
affixing a sealing plate on an opening in said flexible floating membrane;
mounting a flange to said outlet pipe;
mounting lifting hooks to said sealing plate;
hoisting said lifting hooks and said sealing plate to said flange, and
bolting said flange to said sealing plate.

14. A method for fabricating a sheet joint of a segmented floating plastic-based cover for a wastewater reservoir;
overlapping a top layer of said sheet joint over a bottom layer of said sheet joint;
welding said top layer along an edge of said bottom layer, forming a thermoplastic weld through said top and bottom layers;
during said step of welding, leaving a longitudinal flap in said top layer extending away from said thermoplastic weld and said edge of said bottom layer; said longitudinal flap being as wide as a fused portion of said thermoplastic weld and a work-holding margin on each side of said fused portion.
